(19) United States
(12) Patent Application Publication
(10) Pub. No.: US 2025/0288822 A1
(43) Pub. Date: Sep. 18, 2025

(54) CONFIGURABLE PHOTOBIOMODULATION THERAPY

(71) Applicant: Welch Allyn, Inc., Skaneateles Falls, NY (US)

(72) Inventors: Gene J. Wolfe, Pittsford, NY (US); John A. Lane, Venice, FL (US); WonKyung McSweeney, Manlius, NY (US); David E. Quinn, Moravia, NY (US)

(21) Appl. No.: 19/081,079

(22) Filed: Mar. 17, 2025

Related U.S. Application Data
(60) Provisional application No. 63/566,448, filed on Mar. 18, 2024.

(51) Int. Cl. A61N 5/06

(52) U.S. Cl. CPC A61N 5/0616; A61N 2005/0626; A61N 2005/0651; A61N 2005/0663; A61N 2005/0665

(57) ABSTRACT

A device for providing photobiomodulation therapy on a target area of a patient projects a probe beam onto the target area. The device projects a probe beam onto the target area. The device detects anatomical features and properties within the target area based on data collected from the probe beam. The device modulates a therapy beam based on the anatomical features and properties detected within the target area. The device projects the therapy beam onto the target area for providing the photobiomodulation therapy.

### CROSS-REFERENCE TO RELATED APPLICATION

This application claims the benefit of and priority to U.S. Provisional Patent Application No. 63/566,448, filed Mar. 18, 2024, the entire disclosure of which is incorporated by reference herein in its entirety.

### BACKGROUND

Photobiomodulation therapy (PBMT), also known as low-level laser therapy (LLLT) or cold laser therapy, is a non-invasive treatment that uses light to stimulate cellular function and promote healing in a targeted area. PBMT has gained popularity in various medical fields.

PBMT works by delivering photons to the targeted area, which are absorbed by the cells and trigger a series of biochemical reactions. These reactions can lead to increased production of adenosine triphosphate (ATP), the energy currency of cells, and the release of nitric oxide, a molecule involved in vasodilation and anti-inflammatory processes. As a result, PBMT has been found to have several beneficial effects, such as reducing pain and inflammation, accelerating tissue repair, and improving overall tissue function.

The effectiveness of PBMT may vary depending on the condition being treated and the individual receiving the therapy. While some patients experience significant improvements, others may not respond as well or at all. This can be due to the fact that optimal parameters for PBMT, such as wavelength, power density, and treatment duration, are still being researched and investigated. A lack of standardized protocols makes it challenging to compare studies and establish consistent guidelines for PBMT. Consequently, there is a need for further research to better understand the optimal parameters for different conditions and patient populations.

### SUMMARY

In general terms, the present disclosure relates to photobiomodulation therapy. In one possible configuration, one or more parameters of a photobiomodulation therapy beam are modulated based on an analysis of an area targeted by the beam. Various aspects are described in this disclosure, which include, but are not limited to, the following aspects.

One aspect relates to a device for providing photobiomodulation therapy on a target area of a patient, the device comprising: at least one processing device; and at least one computer readable data storage device storing software instructions that, when executed by the at least one processing device, cause the at least one processing device to: project a probe beam onto the target area; detect anatomical features and properties within the target area based on data collected from the probe beam; modulate a therapy beam based on the anatomical features and properties detected within the target area; and project the therapy beam onto the target area for providing the photobiomodulation therapy.

Another aspect relates to a method of modulating photobiomodulation therapy on a target area of a patient, the method comprising: projecting a probe beam onto the target area; detecting anatomical features and properties within the target area based on data collected from the probe beam; modulating a therapy beam based on the anatomical features and properties detected within the target area; and projecting the therapy beam onto the target area for providing the photobiomodulation therapy.

Another aspect relates to a system for configuring a photobiomodulation therapy session, the system comprising: at least one processing device; and at least one computer readable data storage device storing software instructions that, when executed by the at least one processing device, cause the at least one processing device to: aggregate data from a plurality of photobiomodulation therapy sessions; identify one or more parameters for treating a condition in a target area having one or more properties based on the data aggregated from the plurality of photobiomodulation therapy sessions; and generate a treatment plan for the photobiomodulation therapy session, the treatment plan modulating the therapy beam to have the one or more parameters based on the one or more properties detected in the target area.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 illustrates an example of a system for modulating photobiomodulation therapy to improve healing in a target area on a patient.

FIG. 2 schematically illustrates an example of a PBMT device that can be used to perform the photobiomodulation therapy in the target area on the patient of FIG. 1.

FIG. 3 schematically illustrates an example of a method of performing the photobiomodulation therapy that can be performed by the PBMT device of FIG. 2.

FIG. 4 schematically illustrates an example of photobiomodulation therapy parameters that can be modulated by the method of FIG. 3.

FIG. 5 illustrates an example of a therapy light source on the PBMT device of FIG. 2.

FIG. 6 schematically illustrates an example of a method of analyzing data from a probe beam projected by the PBMT device of FIG. 2 to determine anatomical features and properties in the target area on the patient.

FIG. 7 schematically illustrates an example of a probe analysis output generated by the method of FIG. 6.

FIG. 8 schematically illustrates an example of a method of tracking photobiomodulation therapy performed by the PBMT device of FIG. 2.

FIG. 9 schematically illustrates an example of a PBMT analytics system that can be included in the system of FIG. 1.

FIG. 10 schematically illustrates an example of a method of generating PBMT treatment plans that can be performed by the PBMT analytics system of FIG. 9.

### DETAILED DESCRIPTION

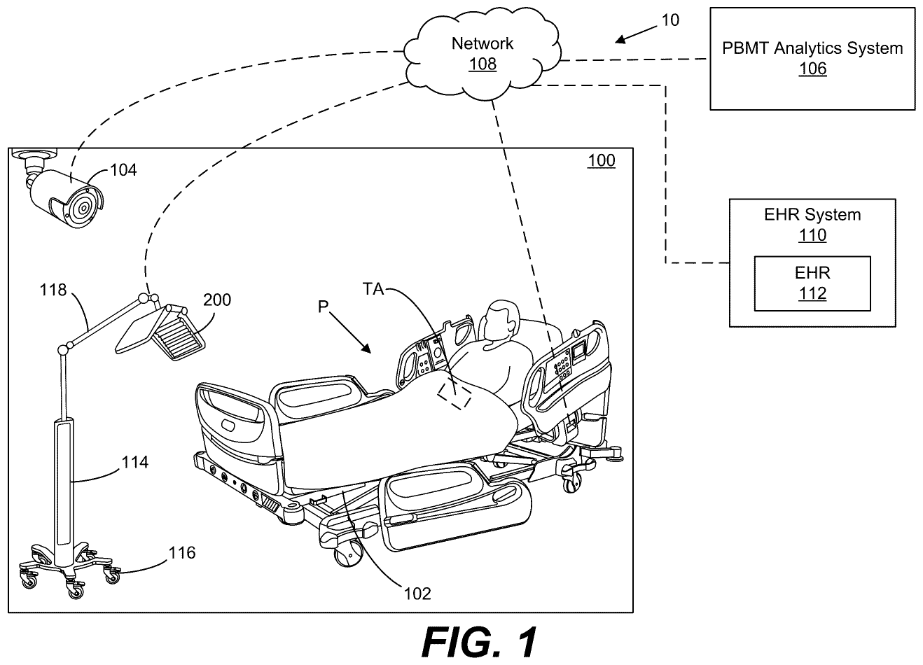

FIG. 1 illustrates an example of a system 10 for modulating photobiomodulation therapy (PBMT) to improve healing in a target area TA on a patient P. In FIG. 1, the patient P is shown inside a patient environment 100. In some examples, the patient environment 100 is a postoperative ward, post operative care unit (POCU), post-surgical unit, a med-surg unit, or other area within a healthcare facility where the patient P is transferred following a surgical procedure.

In the example of FIG. 1, the patient P is shown resting on a patient support apparatus 102 inside the patient environment 100. The patient support apparatus 102 can be a hospital bed, a stretcher, operating room table, or similar type of apparatus on which the patient P can rest. The patient support apparatus 102 can include one or more sensors that measure one or more physiological parameters of the patient P such as heart rate, non-invasive blood pressure (NIBP), patient motion, and patient weight. Additionally, the patient support apparatus 102 can include sensors that detect patient exit, incontinence, deterioration, and other metrics.

The patient environment 100 can include a camera 104 that captures image data of the patient environment 100. For example, the camera 104 can capture video data of the patient P resting on the patient support apparatus 102. The camera 104 can capture video data of medical equipment inside the patient environment 100 and other persons who enter the environment.

The patient environment 100 includes a photobiomodulation therapy (PBMT) device 200 that is used to perform PBMT such as to heal a wound on the patient P that is located inside the target area TA and that can be result of a surgical procedure. In FIG. 1, the target area TA is covered by a blanket that is placed over the patient P. The PBMT device 200 can be positioned under the blanket (and any garment worn by the patient P) such that the target area TA is exposed without obstructions to the PBMT device 200 for photobiomodulation therapy.

In the example of FIG. 1, the PBMT device 200 is illustrated as being mounted on a portable cart 114 that includes casters 116 for moving the PBMT device 200 around the patient environment 100. Further, the portable cart 114 includes an articulated arm 118 that can be used to position the PBMT device 200 over the wound in the target area TA on the patient P.

It is contemplated that the PBMT device 200 can be mounted on pieces of equipment that differ from the portable cart 114 shown in FIG. 1. Further, the PBMT device 200 can have different form factors than the one shown in FIG. 1. Accordingly, the description provided herein is not limited to the example of the PBMT device 200 shown in FIG. 1.

In another alternative example, the PBMT device 200 can be a handheld device that can be positioned by a caregiver or the patient P over the target area TA. As a further illustrative example, the PBMT device 200 can be mounted on a pad or blanket that can be placed over the target area TA. As a further illustrative example, the PBMT device 200 can be mounted on a wearable item such as a patch or a bandage that can be temporarily attached to the patient P over the target area TA. As yet a further example, the PBMT device 200 can be mounted on a helmet worn by the patient P such as when the target area is on the skull of the patient P.

In further examples, the PBMT device 200 can have a form factor that allows it to enter an internal cavity of the patient P such as a mouth, ear, nose, or rectum. In such examples, the PBMT device 200 can be used to promote healing inside the internal cavity of the patient P.

As further shown in FIG. 1, the patient support apparatus 102, the camera 104, and the PBMT device 200 are each communicatively connected to a photobiomodulation therapy (PBMT) analytics system 106 over a network 108. As will be described in more detail, the PBMT analytics system 106 can modulate the photobiomodulation therapy performed by the PBMT device 200 based on one or more parameters that are captured and/or measured from a probe beam emitted by the PBMT device 200. The PBMT analytics system 106 can receive the one or more parameters captured and/or measured from the probe beam over the network 108.

In addition to the one or more parameters captured and/or measured from the probe beam emitted by the PBMT device 200, the PBMT analytics system 106 can also modulate the photobiomodulation therapy performed by the PBMT device 200 based on one or more parameters that are captured and/or measured by the patient support apparatus 102 and/or the camera 104. For example, the PBMT analytics system 106 can modulate the photobiomodulation therapy performed by the PBMT device 200 based on one or more physiological parameters captured by the patient support apparatus 102 such as heart rate, non-invasive blood pressure (NIBP), patient motion, and patient weight. In further examples, the PBMT analytics system 106 can modulate the photobiomodulation therapy performed by the PBMT device 200 based on the image data captured by the camera 104 that can be used to locate the relative positions of the target area TA on the patient P and the PBMT device 200 inside the patient environment 100.

In some examples, the patient support apparatus 102, the camera 104, and the PBMT device 200 are each communicatively connected to an EHR system 110 over the network 108. One or more parameters captured by the patient support apparatus 102, the camera 104, and the PBMT device 200 can be stored in an electronic health record (EHR) 112 of the patient P.

As described herein, the terms electronic medical records (EMRs) and electronic patient record (EPRs) can be used interchangeably with EHRs. The EHR system 110 collects patient electronically stored health information in a digital format (e.g., EHRs 112). As such, the EHR system 110 maintains a plurality of EHRs 112 for a plurality of patients. Each EHR 112 can be shared across different health care settings. For example, the EHRs 112 are shared through network-connected, enterprise-wide information systems or other information networks and exchanges. The EHRs 112 may include a range of data, including demographics, medical history, medication and allergies, immunization status, laboratory test results, radiology images, vital signs, personal statistics like age and weight, and billing information.

In some further examples, the PBMT analytics system 106 can modulate the photobiomodulation therapy performed by the PBMT device 200 based on one or more parameters obtained from the EHR 112 of the patient P such as the patient P's medical history including medical conditions, diagnoses, and medications, as well as the patient's demographic information such as age, gender, and race/ethnicity. In further examples, the PBMT analytics system 106 can modulate the photobiomodulation therapy performed by the PBMT device 200 based on data from prior PBMTs performed on the patient P stored in the EHR 112.

The network 108 can connect and exchange data between the devices inside the patient environment 100 such as between the patient support apparatus 102, the camera 104, and the PBMT device 200. In some examples, the network 108 is an Internet of things (IoT) network. Further, the network 108 can also connect and exchange data between the devices inside the patient environment 100 and systems outside of the patient environment 100 such as the PBMT analytics system 106 and the EHR system 110. The network 108 can include any type of wired or wireless connections, or any combinations thereof. In some examples, the wireless connections can be accomplished using Wi-Fi, ultra-wideband (UWB), Bluetooth, and the like.

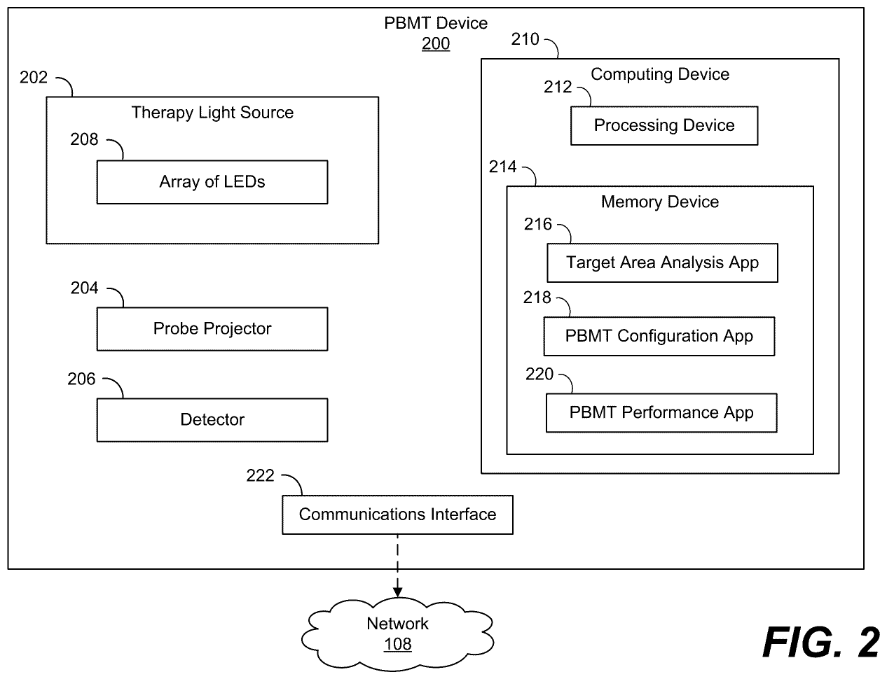

FIG. 2 schematically illustrates an example of the PBMT device 200. As shown in FIG. 2, the PBMT device 200 includes a therapy light source 202 that emits or projects a therapy beam onto the target area TA for healing a wound within the target area. The therapy light source 202 can include an array of light-emitting diodes (LEDs) 208. The array of LEDs 208 can emit light in the red light spectrum (600-810 nm) and/or in the near-infrared light spectrum (800-1064 nm). In some examples, the array of LEDs 208 can emit light in the UVA light spectrum (315-400 nm) and/or the UVB light spectrum (280-315 nm) for providing a therapeutic therapy. One or more parameters of the therapy light source 202 can be controlled to optimize an efficacy of photobiomodulation therapy (PBMT) performed on the target area. For example, an intensity, a wavelength, a duration, an illumination angle, and an illumination location of the array of LEDs 208 can be controlled to optimize the PBMT performed on the target area.

The PBMT device 200 further includes a probe projector 204 and a detector 206. The probe projector 204 and the detector 206 allow the PBMT device 200 and/or the PBMT analytics system 106 to detect anatomical features and properties in the target area TA that can affect photobiomodulation therapy efficacy. For example, the probe projector 204 emits a probe beam onto the target area, and the detector 206 collects data from target area that is analyzed to detect anatomical features and properties such as location of a wound or abrasion, collagen content of skin, thickness of the epidermis, dermis, and other tissue layers, and moisture level. In examples where the PBMT device 200 is mounted on a helmet worn by the patient P, the detector 206 can detect a thickness and a shape of the skull which may influence the efficacy of PBMT.

The PBMT device 200 and/or the PBMT analytics system 106 use the anatomical features and properties identified in the target area to control the therapy light source 202 to modulate the therapy beam projected onto the target area to improve efficacy of the PBMT. Also, the PBMT analytics system 106 uses the anatomical features and properties identified in the target area to track of the efficacy of PBMT on the patient P to improve PBMT treatment plans that are tailored based on presence of anatomical features and properties.

In one example, the probe projector 204 emits the probe beam onto the target area, and the detector 206 detects primary and secondary scatter in the target area from the probe beam. The probe beam is controlled and modulated to obtain data on anatomical features and properties within the target area. For example, an intensity of the probe beam can be controlled to increase and decrease at various levels to capture data at various depths of tissue in the target area. In some examples, the detector 206 includes a photodiode, an avalanche photodiode (APD), or a photomultiplier tube for collecting the scattered light that results from the probe beam.

In some examples, the probe projector 204 emits the probe beam to have a structured illumination pattern that maps onto a textured surface in the target area of the patient. In further examples, the probe projector 204 emits the probe beam to illuminate the target area with color balanced light that provides a high dynamic range in the tissues illuminated by the probe beam.

In some examples, the detector 206 includes a camera that captures images of the target area when illuminated by the probe beam emitted by the probe projector 204. In some examples, the detector 206 is coaxially aligned with the probe projector 204 on the PBMT device 200 to collect the images of the target area illuminated by the probe beam.

In some examples, the probe projector 204 controls and modulates the probe beam based on one or more features or properties of the target area. As an illustrative example, the probe projector 204 controls and modulates the probe beam to have a lower color temperature (e.g., 2300 Kelvin (K)) when the target area is the ear of the patient P. As another illustrative example, the probe projector 204 controls and modulates the probe beam to have a higher color temperature (e.g., 6500 K) when the target area is a skin surface on the patient P's body.

In some examples, the probe beam is in the same spectral domain as the therapy beam. For example, the probe beam can be within the red light spectrum (600-810 nm) and/or in the near-infrared light spectrum (800-1064 nm). In some examples, the detector 206 detects the anatomical features and properties of the target area based on the therapy beam emitted by the therapy light source 202. In such examples, the probe projector 204 can be eliminated from the PBMT device 200 because the therapy beam emitted by the therapy light source 202 can be used to probe the target area, and thereafter the therapy beam can be modulated based on the features and properties of the target area that are detected by the detector 206.

As further shown in FIG. 2, the PBMT device 200 includes a computing device 210 having at least one processing device 212 and a memory device 214. The at least one processing device 212 is an example of a processing unit such as a central processing unit (CPU). The at least one processing device 212 can include one or more central processing units (CPUs). In some examples, the at least one processing device 212 includes one or more digital signal processors, field-programmable gate arrays, and/or other types of electronic circuits.

The memory device 214 operates to store data and instructions for execution by the at least one processing device 212 of the PBMT device 200. In the example illustrated in FIG. 2, the memory device 214 stores a target area analysis application 216, a photobiomodulation therapy (PBMT) configuration application 218, and a photobiomodulation therapy (PBMT) performance application 220, which are described in more detail further below.

The memory device 214 includes computer-readable media, which may include any media that can be accessed by the at least one processing device 212. For example, computer-readable media includes computer readable storage media and computer readable communication media. As such, the memory device 214 is an example of a computer readable data storage device storing software instructions for execution by the at least one processing device 212.

Computer readable storage media includes volatile and nonvolatile, removable and non-removable media implemented in any device configured to store information such as computer readable instructions, data structures, program modules, or other data. Computer readable storage media can include, but is not limited to, random access memory, read only memory, electrically erasable programmable read only memory, flash memory, and other memory technology, including any medium that can be used to store information that can be accessed by the processing device. The computer readable storage media is non-transitory.

Computer readable communication media embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are within the scope of computer readable media.

The PBMT device 200 further includes a communications interface 222 that allows the PBMT device 200 to connect to the network 108. The communications interface 222 can include wired interfaces and/or wireless interfaces. For example, the communications interface 222 can wirelessly connect to the network 108 through Wi-Fi, or other wireless connections. Alternatively, the communications interface 222 can connect to the network 108 using wired connections such as through an Ethernet or Universal Serial Bus (USB) cable.

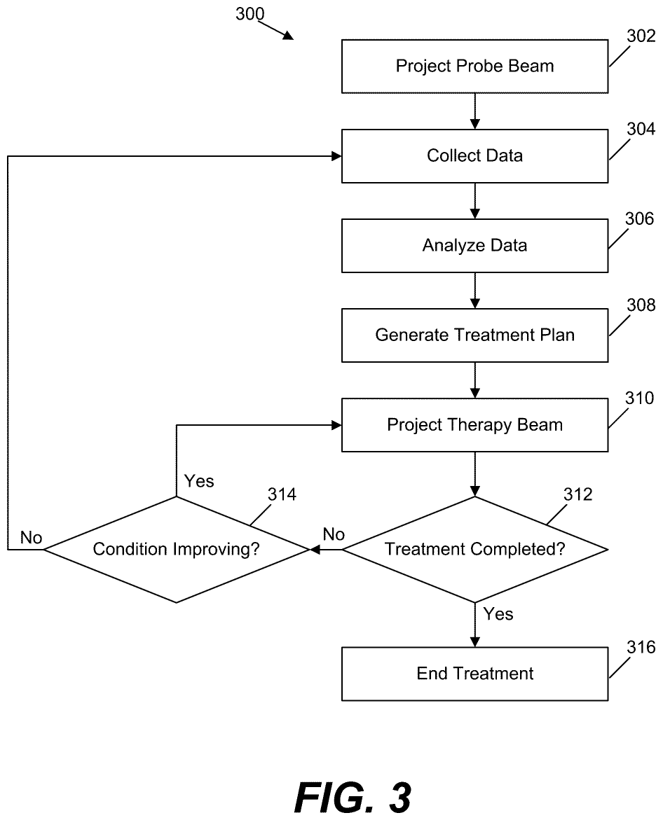

FIG. 3 schematically illustrates an example of a method 300 of performing photobiomodulation therapy (PBMT) that can be performed by the PBMT device 200. The method 300 includes an operation 302 of projecting the probe beam onto the target area. As described above, the probe beam is projected onto the target area by the probe projector 204. The probe beam can be modulated to improve detection of anatomical features such as to increase and decrease an intensity of the probe beam to reach different tissue layers within the target area. In some examples, the probe beam is modulated based on an anatomy of the target area.

In some examples, the therapy beam can be used to probe the target area. In such examples, operation 302 can include projecting the therapy beam onto the target area (instead of emitting a separate probe beam). In such examples, operation 302 can include controlling the therapy light source 202 to project the therapy beam onto the target area.

The method 300 includes an operation 304 of collecting data from the target area while the probe beam is projected onto the target area. Operation 304 can be performed by the detector 206 on the PBMT device 200. In some examples, operations 302 and 304 can occur substantially at the same time such that the operations are simultaneous.

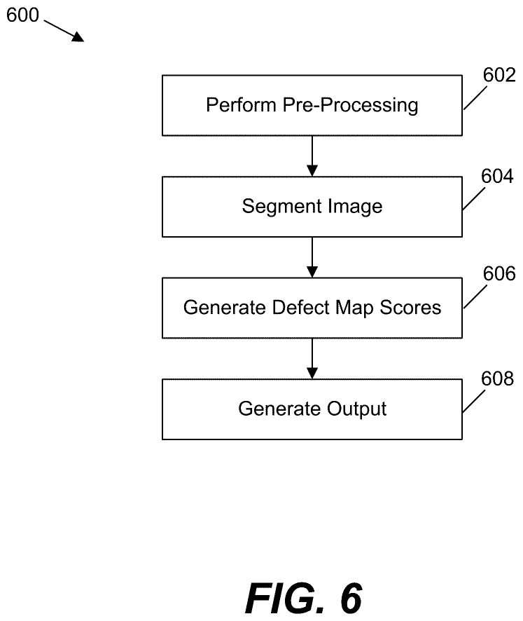

The method 300 includes an operation 306 of analyzing data collected from the target area. Operation 306 can be performed by the target area analysis application 216. In some examples, operation 306 includes aspects of the method 600 described with reference to FIG. 6.

Operation 306 can include analyzing light scatter collected by the detector 206 from the projection of the probe beam on the target area. The light scatter is analyzed in operation 306 to detect anatomical features and properties within the target area such as a location of a wound or abrasion, collagen content of skin, thickness of the epidermis, dermis, and other tissue layers, moisture level, and other properties relevant to the efficacy of photobiomodulation therapy.

In some examples, operation 306 includes analyzing images captured by the detector 206 such as when the probe beam includes a structured illumination pattern that maps onto a 3D textured surface within the target area of the patient, or when the probe beam illuminates the target area with color balanced light that provides a high dynamic range in the tissues.

Operation 306 can include performing artificial intelligence including machine learning algorithms to identify anatomical features and properties based on the data collected from the target area. In some examples, the PBMT device 200 transfers the data collected in operation 304 to the PBMT analytics system 106, and the PBMT analytics system 106 performs algorithms such as artificial intelligence and machine learning algorithms to identify anatomical features and properties based on the data collected from the target area. In such instances, the target area analysis application 216 is performed on the PBMT analytics system 106.

Still referring to FIG. 3, the method 300 includes an operation 308 of generating a treatment plan based on the anatomical features and properties identified in operation 306. The treatment plan modulates one or more parameters of the therapy beam based on the anatomical features and properties to improve the efficacy of the photobiomodulation therapy provided by the PBMT device 200 on the target area of the patient P. In some examples, the treatment plan is generated by the PBMT configuration application 218 performed on the PBMT device 200.

In examples where the PBMT device 200 transfers the data collected in operation 304 to the PBMT analytics system 106 and the PBMT analytics system 106 performs one or more algorithms to identify anatomical features and properties based on the data collected from the target area, operation 308 can also be performed on the PBMT analytics system 106. In such instances, the PBMT configuration application 218 is performed on the PBMT analytics system 106. In such examples, the PBMT analytics system 106 selects a treatment plan for modulating the therapy beam projected by the therapy light source 202 of the PBMT device 200, and communicates the treatment plan to the PBMT device 200 over the network 108.

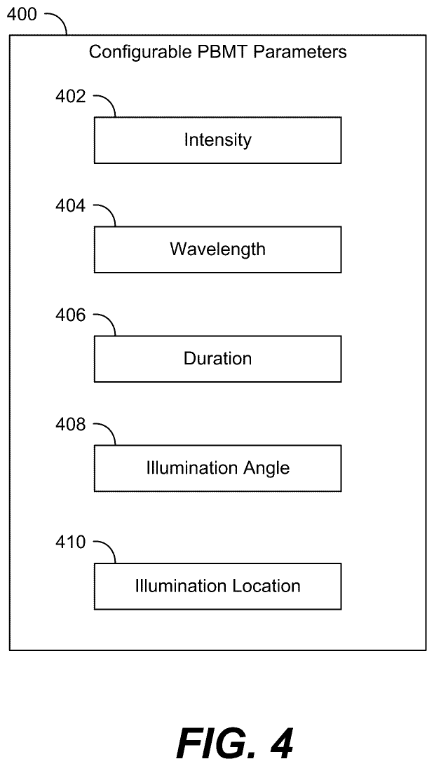

FIG. 4 schematically illustrates example parameters that can be modulated by the treatment plan generated in operation 308. The treatment plan can modulate an intensity 402 of the therapy beam emitted by the therapy light source 202 of the PBMT device 200. For example, the treatment plan can increase the intensity 402 of the therapy beam based on anatomical properties detected in operation 306 such as collagen content of skin, thickness of the epidermis, dermis, and other tissue layers, and moisture level. Alternatively, the treatment plan can decrease the intensity 402 of the therapy beam based on the anatomical properties such as collagen content of skin, thickness of the epidermis, dermis, and other tissue layers, and moisture level.

The treatment plan can also modulate a wavelength 404 of the therapy beam emitted by the therapy light source 202 of the PBMT device 200. For example, the wavelength 404 of the therapy beam can be adjusted within the red light spectrum (600-810 nm) or the near-infrared light spectrum (800-1064 nm) based on the anatomical properties such as collagen content of skin, thickness of the epidermis, dermis, and other tissue layers, and moisture level.

The treatment plan can modulate a duration 406 of the therapy beam emitted by the therapy light source 202 of the PBMT device 200. For example, the duration 406 that the therapy beam is projected by the PBMT device 200 onto the target area can be shortened or can be lengthened based on the anatomical properties detected in operation 306 such as collagen content of skin, thickness of the epidermis, dermis, and other tissue layers, and moisture level.

The treatment plan can modulate an illumination angle 408 of the therapy beam emitted by the therapy light source 202 of the PBMT device 200 based on the anatomical properties such as the collagen content of skin, thickness of the epidermis, dermis, and other tissue layers, and moisture level. For example, an angle of incidence of the therapy beam on the target area can be modulated to be 90 degrees, or can be modulated to be less than 90 degrees, to improve efficacy of the photobiomodulation therapy based on the anatomical properties.

In further examples, the treatment plan can modulate an illumination location 410 of the therapy beam emitted by the therapy light source 202 of the PBMT device 200 based on anatomical landmarks and features detected in operation 306. For example, a portion of the array of LEDs 208 in the therapy light source 202 can be illuminated to target an anatomical feature such as a wound or an abrasion in the target area, while other portions of the array of LEDs 208 are turned off such that therapy beam is focused on treating the wound or abrasion.

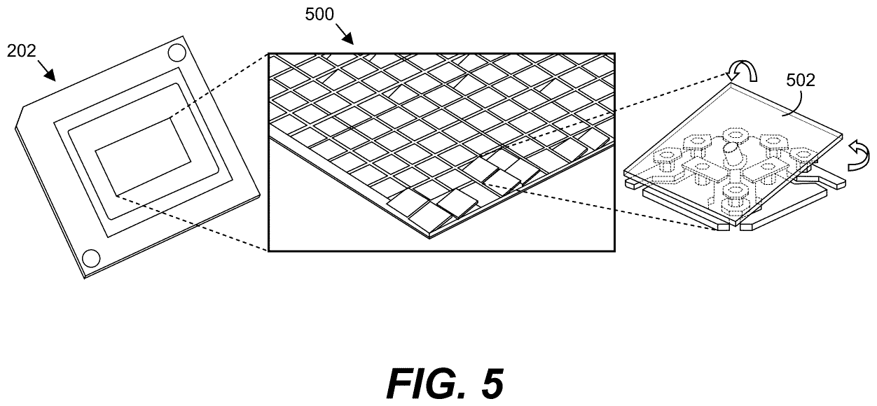

FIG. 5 schematically illustrates an example of the therapy light source 202 on the PBMT device 200. In this example, the therapy light source 202 includes a digital micromirror device (DMD) 500 that can be used to control the illumination location 410 of the therapy beam emitted by the array of LEDs 208. The DMD 500 is an optical micro-electrical-mechanical system (MEMS) that contains an array of pixels 502. As an illustrative example, the pixels 502 are electro-mechanical elements that can include reflective aluminum micromirrors.

The pixels 502 can be individually rotated (e.g., +/−10-12 degrees) to an on-state or an off-state. In the on-state, light from the LEDs of the therapy light source 202 is reflected into a lens for projection onto the target area. In the off-state, the light is directed elsewhere (e.g., a heatsink) such that the light is not projected onto the target area. In some examples, the illumination angle 408 and the illumination location 410 of the light emitted by the array of LEDs 208 are modulated by individually controlling the pixels 502 of the DMD 500.

Referring back to FIG. 3, the method 300 includes an operation 310 of projecting the therapy beam onto the target area based on the treatment plan generated in 308. The therapy beam is projected by the array of LEDs 208 of the therapy light source 202.

The method 300 can further include an operation 312 of determining whether the PBMT treatment is completed. In operation 312, determining whether the PBMT treatment is completed can be based on the treatment plan generated in operation 308. For example, completion of the PBMT treatment is based on the duration 406 of the therapy beam. In further examples, operation 312 can include determining whether the PBMT treatment is completed based on the anatomical features and properties determined from the data captured by the detector 206 of the PBMT device 200, which can include data from the probe beam emitted by the probe projector 204 and/or the therapy beam emitted by the therapy light source 202.

When operation 312 determines the treatment is complete (i.e., “Yes” in operation 312), the method 300 proceeds to an operation 316 of ending the PBMT treatment. When operation 312 determines the treatment is not complete (i.e., “No” in operation 312), the method 300 proceeds to an operation 314 of determining whether the condition is improving.

Operation 314 can include analyzing data collected from the target area when the probe beam is projected on the target area by the probe projector 204. Alternatively, operation 314 can include analyzing data collected from the target area based on the therapy beam that is projected on the target area in operation 310. Operation 314 can include determining whether a size of a wound or abrasion has decreased, whether moisture level has decreased, or whether some other type of characteristic in the target region indicates improvement of the condition.

When operation 314 determines that the condition in the target area is improving (i.e., “Yes” in operation 314), the method 300 continues projecting the therapy beam in operation 310 based on the treatment plan generated in operation 308. Otherwise, when operation 314 determines that the condition in the target area is not improving (i.e., “No” in operation 314), the method 300 repeats the operations 302-310 such that a new therapy beam is projected in repetition of operation 310 based on a new treatment plan generated in repetition of operation 308. The new therapy beam can be modulated such as to increase the intensity 402 and the duration 406 of the therapy beam to provide a stronger dosage of the PBMT. Additionally, the wavelength 404, the illumination angle 408, and/or the illumination location 410 can be adjusted to improve the efficacy of the photobiomodulation therapy provided by the PBMT device 200. In this manner, the method 300 includes updating the therapy beam to improve efficacy of the photobiomodulation therapy while it is being performed on the patient P.

FIG. 6 schematically illustrates an example of a method 600 of analyzing data from the probe beam projected by the PBMT device 200 to determine anatomical features and properties in the target area. In some instances, the method 600 is performed by the target area analysis application 216 which can be performed on the PBMT device 200 or can be performed on the PBMT analytics system 106. In some examples, the method 600 is performed as operation 306 of analyzing data collected from the target area in the method 300 shown in FIG. 3.

The method 600 analyzes data collected from the target area resulting from the probe beam emitted by the probe projector 204. The probe beam can be emitted as a beam separate from the therapy beam emitted by the therapy light source 202. In alternative examples, the therapy beam emitted by the therapy light source 202 can be analyzed by the method 600 for determining the anatomical features and properties in the target area.

The method 600 can include an operation 602 of performing pre-processing on an image captured by the detector 206. As described above, in some examples the detector 206 is a camera that captures images of the target area when illuminated by the probe beam emitted by the probe projector 204 or when illuminated by the therapy beam emitted by the therapy light source 202. Operation 602 can include removing aberrations from the images of the target area that can result from defective pixels, movement artefacts, lens non-uniformity, and the like. Operation 602 can also include removing external obstructions from the image including spittle, blood, and/or other bodily fluid that is obstructing a view of the target area.

The method 600 can include an operation 604 of segmenting the image into regions based on anatomical landmarks in the target area. Operation 604 can include labeling the regions, and then connecting the regions to form an atlas on the image captured by the detector 206.

The method 600 can include an operation 606 generating defect map scores for each region segmented in operation 604. The defect map scores are calculated based on a clinical condition in the target area and how the clinical condition presents in the characteristics of the image. The clinical condition in the target area can be identified from the EHR 112 of the patient P (see FIG. 1). As an illustrative example, when the condition is a wound from a surgical procedure, operation 606 can include generating defect map scores based on loss of color, inflammation, blood, scabs, and other characteristics related to wound healing. The defect map scores allow collection of a set of images that are representative of the target area.

The method 600 includes an operation 608 of generating an output based on the defect map scores that are generated in operation 606. The output generated in operation 608 can include collected images of the target area defect maps that are knitted, fused, or stitched into a final image data set. The output generated in operation 608 is used to modulate the therapy beam projected by the therapy light source 202 to improve efficacy of the photobiomodulation therapy performed on the target area of the patient P.

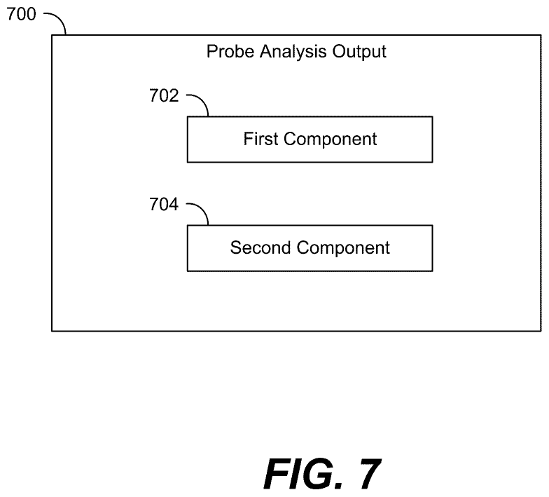

FIG. 7 schematically illustrates an example of a probe analysis output 700 generated in operation 608 of the method 600. The probe analysis output 700 includes a first component 702 that identifies a treatment location in 3-dimensional space to direct the therapy beam emitted by the array of LEDs 208 of the therapy light source 202. The first component 702 that includes the treatment location is used to focus the therapy beam on a particular anatomical feature such as a wound or abrasion in the target area, and to ignore other portions in the target area.

The probe analysis output 700 includes a second component 704 that identifies properties of the anatomical feature in the target area. The anatomical properties can include collagen content of skin, thickness of the epidermis, dermis, and other tissue layers, moisture level, and other properties relevant to the condition of the anatomical feature in the target area.

The probe analysis output 700 is fed to the PBMT configuration application 218 that computes an output profile for the therapy beam for emission by the therapy light source 202. The PBMT configuration application 218 can be performed on the PBMT device 200, or alternatively can be performed on the PBMT analytics system 106.

The output profile generated by the PBMT configuration application 218 focuses the therapy beam based on the first component 702 of the probe analysis output 700 by controlling the pixels 502 of the DMD 500 to rotate between the on-state and the off-state. As described above, in the on-state, light from the array of LEDs 208 is projected onto the target area, whereas in the off-state, the light is not projected onto the target area. The output profile further modulates the therapy beam based on the second component 704 of the probe analysis output 700 by modulating one or more of the intensity 402, the wavelength 404, the duration 406, the illumination angle 408, and the illumination location 410 of the therapy beam.

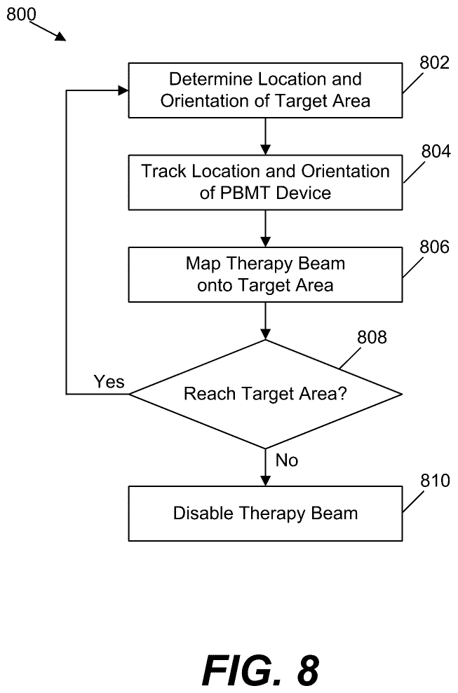

FIG. 8 schematically illustrates an example of a method 800 of tracking the PBMT performed by the PBMT device 200 on the target area of the patient. The method 800 can be performed by the PBMT performance application 220 on the PBMT device 200. The method 800 can be especially useful in examples where the PBMT device 200 is handheld such that a location and orientation of the PBMT relative to the target area may vary while the PBMT device 200 is being held by a caregiver or the patient P to perform the PBMT on the target area.

The method 800 includes an operation 802 of determining a location and orientation of the target area. Operation 802 can be performed by using the probe projector 204 to emit the probe beam and using the detector 206 to detect light scatter. In this example, the light scatter can be analyzed to detect the location and orientation of the target area on the patient P.

The method 800 includes an operation 804 of tracking a location and orientation of the PBMT device 200. Operation 804 can be performed by analyzing the light scatter from the probe beam to determine the location and orientation of the PBMT device 200. Alternatively, operation 804 can be performed by analyzing the image data collected by the camera 104 (see FIG. 1) to determine the location and orientation of the PBMT device 200.

The method 800 includes an operation 806 of mapping the therapy beam onto the target area based on the location and orientation of the PBMT device 200 (determined in operation 804) relative to the location and orientation of the target area (determined in operation 802). Operation 806 can include computing a set of vectors that map the therapy beam onto the target area by dynamically modulating the therapy beam profile, as described above.

The method 800 can include an operation 808 of determining whether the therapy beam is able to reach the target area. When the therapy beam is determined as not being able to reach the target area on the patient P (i.e., “No” in operation 808), the method 800 proceeds to an operation 810 of disabling the therapy beam. Operation 810 can include generating an alert to notify the caregiver or the patient P that the therapy beam is disabled because it is not able to reach the target area such that the caregiver or the patient P should reposition the PBMT device 200 to allow the therapy beam to reach the target area. In this manner, the method 800 conserves energy use by the PBMT device 200 and improves efficacy of the PBMT device 200 by ensuring that the therapy beam is generated only when able to reach the target area.

As shown in FIG. 8, when the therapy beam is determined as being able to reach the target area on the patient P (i.e., “Yes” in operation 808), the method 800 repeats the operations 802-808 such that the method 800 continuously optimizes the profile of the therapy beam emitted by the therapy light source 202 based on the location and orientation of the PBMT device 200 relative to the target area, which can dynamically change due to movements by the patient P and/or the PBMT device 200, especially when the PBMT device 200 is handheld.

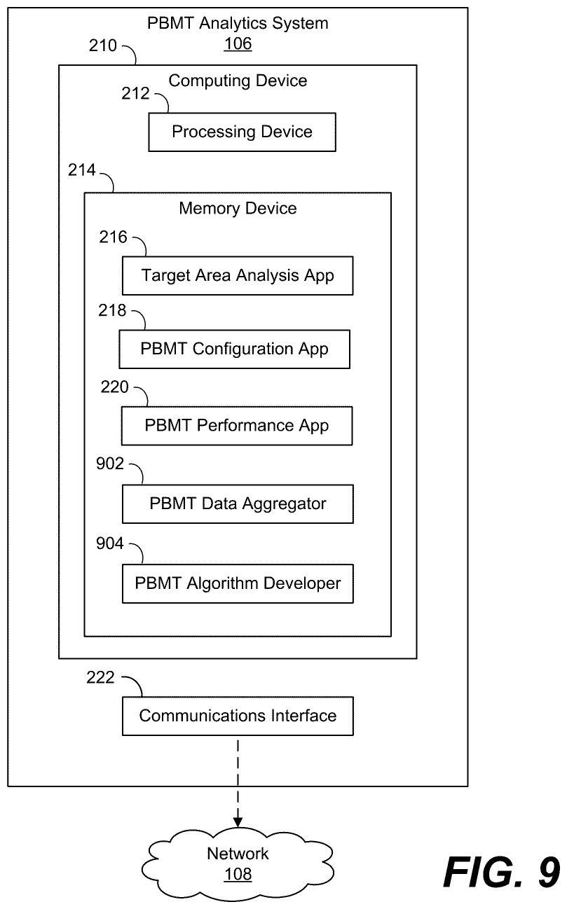

FIG. 9 schematically illustrates an example of the PBMT analytics system 106. As shown in FIG. 9, the PBMT analytics system 106 includes a computing device 210 having at least one processing device 212 and a memory device 214. The PBMT analytics system 106 further includes a communications interface 222 that allows the PBMT analytics system 106 to connect to the network 108. The computing device 210, the at least one processing device 212, the memory device 214, and the communications interface 222 are similar to the devices of the PBMT device 200 that are described above such that the descriptions of these devices with respect to the PBMT device 200 can similarly apply to the PBMT analytics system 106.

In the example shown in FIG. 9, the memory device 214 of the PBMT analytics system 106 stores the target area analysis application 216, the PBMT configuration application 218, and the PBMT performance application 220, which are described above. Aspects of the target area analysis application 216, the PBMT configuration application 218, and the PBMT performance application 220 can be performed on the PBMT analytics system 106 in addition or as an alternative to performing these applications on the PBMT device 200.

The memory device 214 of the PBMT analytics system 106 further stores a photobiomodulation therapy (PBMT) data aggregator 902 and a photobiomodulation therapy (PBMT) algorithm developer 904. The PBMT data aggregator 902 aggregates data from a plurality of PBMT sessions performed by the PBMT device 200 on the patient P and on different patients as well. Also, the PBMT data aggregator 902 aggregates data from PBMT sessions performed by a plurality of PBMT devices 200 connected to the network 108. The data from the PBMT sessions is received by the PBMT analytics system 106 over the network 108, and can be aggregated in a databased maintained by the PBMT analytics system 106.

The PBMT algorithm developer 904 uses the aggregated data from the PBMT sessions to generate output profiles for the therapy beam emitted by the therapy light source 202 of the PBMT device 200. Additionally, the PBMT algorithm developer 904 continuously updates the output profiles as new data is aggregated by the PBMT data aggregator 902.

In some examples, aspects of the PBMT data aggregator 902 and the PBMT algorithm developer 904 are performed on the PBMT device 200. For example, the PBMT device 200 can aggregate PBMT data to continuously update the output profiles for optimal emission of the therapy beam by the therapy light source 202.

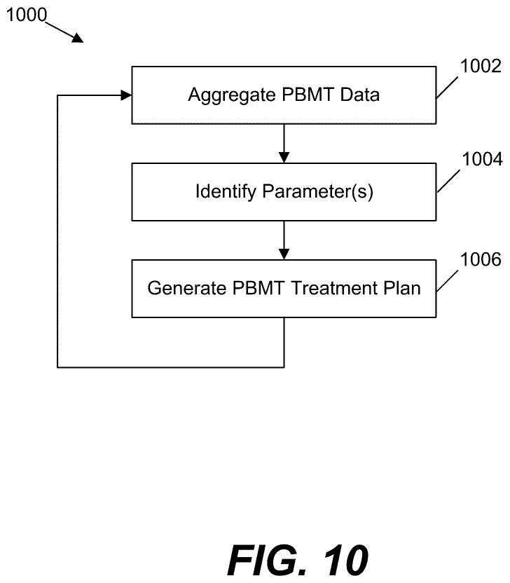

FIG. 10 schematically illustrates an example of a method 1000 of generating PBMT treatment plans. The method 1000 can be performed by the PBMT algorithm developer 904 on PBMT analytics system 106. Alternatively, aspects of the method 1000 can be performed by the PBMT algorithm developer 904 when executed on the PBMT device 200.

The method 1000 includes an operation 1002 of aggregating data from a plurality of PBMT sessions. The data can include the output profiles of the therapy beams projected by the PBMT device 200. The output profiles can include one or more properties of the therapy beams such as one or more of the intensity 402, the wavelength 404, the duration 406, the illumination angle 408, and the illumination location 410 of the therapy beam.

The data aggregated in operation 1002 can further include features and properties detected in areas targeted by the therapy beams such as one or more diseases, conditions, types of wounds or abrasions detected in the areas, and properties detected in the areas such as collagen content, thickness of the epidermis, dermis, and other layers, and moisture level.

The data aggregated in operation 1002 can further include outcomes from the plurality of PBMT sessions. The outcomes can include scores that evaluate the efficacy of the PBMT sessions such as whether a PBMT session was effective in treating a disease, condition, type of wound or abrasion in an area targeted by a therapy beam. As an example, the scores quantify a level healing of a wound or an abrasion in the target area such as whether a size of the wound or abrasion decreased, or whether a moisture level detected in the target area decreased.

The method 1000 includes an operation 1004 of identifying a parameter or a combination of parameters in the output profiles of the therapy beams that are most effective in treating a condition in a target area having a property or a combination of properties. As an illustrative example, operation 1004 can include identifying an intensity, a wavelength, a duration, an illumination angle, and an illumination location, whether individually or in combination with one another, that is most effective for treating a condition in a target area having one or more properties such as collagen content, thickness of the epidermis, dermis, and other layers, and moisture level. In some instances, operation 1004 can be performed by artificial intelligence and/or machine learning algorithms executed on the PBMT analytics system 106.

The method 1000 includes an operation 1006 of generating a PBMT treatment plan based on the parameter or the combination of parameters identified in operation 1004. Operation 1006 can include generating a PBMT treatment plan that modulates one or more parameters such as the intensity 402, the wavelength 404, the duration 406, the illumination angle 408, and the illumination location 410 for optimally treating a condition in a target area having one or more properties such as collagen content, thickness of the epidermis or dermis, and moisture level.

The PBMT treatment plan generated in operation 1006 can be stored in a memory such as the memory device 214 of the PBMT device 200 or the memory device 214 of the PBMT analytics system 106. Accordingly, when a probe analysis output 700 is received, the probe analysis output 700 can be matched to a pre-generated PBMT treatment plan that modulates the therapy beam for optimal efficacy based on the components in the probe analysis output 700.

The various embodiments described above are provided by way of illustration only and should not be construed to be limiting in any way. Various modifications can be made to the embodiments described above without departing from the true spirit and scope of the disclosure.

What is claimed is:

1. A device for providing photobiomodulation therapy on a target area of a patient, the device comprising:
at least one processing device; and
at least one computer readable data storage device storing software instructions that, when executed by the at least one processing device, cause the at least one processing device to:
project a probe beam onto the target area;
detect anatomical features and properties within the target area based on data collected from the probe beam;
modulate a therapy beam based on the anatomical features and properties detected within the target area; and
project the therapy beam onto the target area for providing the photobiomodulation therapy.

2. The device of claim 1, wherein modulate the therapy beam includes adjusting at least one of an intensity, a wavelength, a duration, an illumination angle, and an illumination location.

3. The device of claim 1, wherein the anatomical features and properties detected within the target area include at least one of a location of a wound or an abrasion, collagen content of skin, thickness of an epidermis layer or a dermis layer, and moisture level.

4. The device of claim 1, further comprising:
a light source having an array of light-emitting diodes for emitting the therapy beam.

5. The device of claim 1, wherein the therapy beam is emitted in the red light spectrum or in the near-infrared light spectrum.

6. The device of claim 1, wherein the instructions, when executed by the at least one processing device, further cause the imaging device to:
determine whether a condition in the target area is improving based on the data collected from the target area; and
modulate one or more parameters of the therapy beam when the condition in the target area is not improving.

7. The device of claim 1, wherein the instructions, when executed by the at least one processing device, further cause the imaging device to:
determine a location and an orientation of the target area;
track a location and an orientation of a light source having an array of light-emitting diodes for emitting the therapy beam; and
map the therapy beam onto the target area based on the location and the orientation of the light source relative to the location and the orientation of the target area.

8. The device of claim 7, wherein the instructions, when executed by the at least one processing device, further cause the imaging device to:
determine whether the therapy beam reaches the target area; and
disable the therapy beam when the therapy beam does not reach the target area.

9. A method of modulating photobiomodulation therapy on a target area of a patient, the method comprising:
projecting a probe beam onto the target area;
detecting anatomical features and properties within the target area based on data collected from the probe beam;
modulating a therapy beam based on the anatomical features and properties detected within the target area; and
projecting the therapy beam onto the target area for providing the photobiomodulation therapy.

10. The method of claim 9, wherein modulating the therapy beam includes adjusting at least one of an intensity, a wavelength, a duration, an illumination angle, and an illumination location.

11. The method of claim 9, wherein the anatomical features and properties detected within the target area include at least one of a location of a wound or an abrasion, collagen content of skin, thickness of an epidermis layer or a dermis layer, and moisture level.

12. The method of claim 9, wherein the therapy beam is emitted in the red light spectrum or in the near-infrared light spectrum.

13. The method of claim 9, further comprising:
determining whether a condition in the target area is improving based on the data collected from the target area; and
modulating one or more parameters of the therapy beam when the condition in the target area is not improving.

14. The method of claim 9, further comprising:
determining a location and an orientation of the target area;
tracking a location and an orientation of a light source having an array of light-emitting diodes for emitting the therapy beam; and
mapping the therapy beam onto the target area based on the location and the orientation of the light source relative to the location and the orientation of the target area.

15. The method of claim 9, further comprising:
determining whether the therapy beam reaches the target area; and
disabling the therapy beam when the therapy beam does not reach the target area.

16. A system for configuring a photobiomodulation therapy session, the system comprising:
at least one processing device; and
at least one computer readable data storage device storing software instructions that, when executed by the at least one processing device, cause the at least one processing device to:
aggregate data from a plurality of photobiomodulation therapy sessions;
identify one or more parameters for treating a condition in a target area having one or more properties based on the data aggregated from the plurality of photobiomodulation therapy sessions; and
generate a treatment plan for the photobiomodulation therapy session, the treatment plan modulating the therapy beam to have the one or more parameters based on the one or more properties detected in the target area.

17. The system of claim 16, where the treatment plan modulates at least one of an intensity, a wavelength, a duration, an illumination angle, and an illumination location of the therapy beam.

18. The system of claim 16, wherein the one or more properties detected in the target area include at least one of a location of a wound or an abrasion, collagen content of skin, thickness of an epidermis layer or a dermis layer, and moisture level.

19. The system of claim 16, wherein the data aggregated from the plurality of photobiomodulation therapy sessions includes output profiles of therapy beams, properties detected in areas targeted by the therapy beams, and outcomes of the plurality of photobiomodulation therapy sessions.

20. The system of claim 16, wherein the one or more parameters are identified by artificial intelligence.
